(19) United States
(12) Patent Application Publication
(10) Pub. No.: US 2013/0066189 A1
(43) Pub. Date: Mar. 14, 2013

(54) NORMATIVE DATASET FOR NEUROPSYCHIATRIC DISORDERS

(71) Applicants: Lyubomir Georgiev Zagorchev, Burlington, MA (US); Reinhard Kneser, Aachen (DE); Dieter Geller, Aachen (DE); Yuechen Qian, Briarcliff Manor, NY (US); Juergen Weese, Aachen (DE); Matthew A. Garlinghouse, Hanover, NH (US); Robert M. Roth, Lebanon, NH (US); Thomas W. McAllister, Wilmot, NH (US)

(72) Inventors: Lyubomir Georgiev Zagorchev, Burlington, MA (US); Reinhard Kneser, Aachen (DE); Dieter Geller, Aachen (DE); Yuechen Qian, Briarcliff Manor, NY (US); Juergen Weese, Aachen (DE); Matthew A. Garlinghouse, Hanover, NH (US); Robert M. Roth, Lebanon, NH (US); Thomas W. McAllister, Wilmot, NH (US)

(73) Assignee: KONINKLIJKE PHILIPS ELECTRONICS N.V., Eindhoven (NL)

(21) Appl. No.: 13/580,390

(22) PCT Filed: Feb. 2, 2011
(86) PCT No.: PCT/IB2011/050450

§ 371 Date: Nov. 28, 2012

Related U.S. Application Data
(60) Provisional application No. 61/309,543, filed on Mar. 2, 2010.

(51) Int. Cl. A61B 6/00

(52) U.S. Cl. 600/407

(57) ABSTRACT

A system and method for identifying an abnormality of an anatomical structure. The system and method segments, using a processor, the anatomical structure imaged in a volumetric image of a plurality of control patients to produce a control segmentation of the anatomical structures of each of the control patients, obtains a normative dataset by extracting a statistical representation of a morphology of the control segmentations, segments the anatomical structure of a patient being analyzed for abnormalities to produce a patient segmentation and compares the patient segmentation to the normative dataset obtained from the control segmentations.

### BACKGROUND

Many common neuropsychiatric disorders (e.g., Alzheimer's, schizophrenia, depression) may represent a number of different disorders that appear clinically similar, but respond differently to treatment. These underlying differences may reflect variable disease specific neural substrates. Thus, rapid identification of volumetric and shape abnormalities of specific brain areas relevant to the neuropathophysiology of such disorders would be helpful in characterizing disease subtypes and would most likely improve therapeutic outcomes. Identifying individuals with psychiatric and neurological disorders before the full onset of the symptoms of the disorders could allow for early intervention strategies aimed at preventing onset altogether and/or improving its long-term course.

Currently, decisions about morphology of brain structures in most clinical centers are restricted to subjective review of MRI images because of the labor-intensive nature of manual parcellation of MRI brain volumes and the lack of highly accurate and time efficient automatic tools. In addition, physicians are often concerned with a single brain structure at a time. However, the brain is an interconnected network of tissues. Thus, the investigation of multiple structures simultaneously may reveal important information that has the potential to shed new insights to important questions.

### SUMMARY OF THE INVENTION

A method for identifying an abnormality of an anatomical structure by comprising segmenting, using a processor, the anatomical structure imaged in a volumetric image of a plurality of control patients to produce a control segmentation of the anatomical structures of each of the control patients, obtaining a normative dataset by extracting a statistical representation of a morphology of the control segmentations, segmenting the anatomical structure of a patient being analyzed for abnormalities to produce a patient segmentation, and comparing the patient segmentation to the normative dataset obtained from the control segmentations.

A system for identifying abnormalities of an anatomical structure having a processor segmenting the anatomical structure imaged in a volumetric image of a plurality of control patients to produce a control segmentation of the anatomical structures of each of the control patients and obtaining a normative dataset by extracting a statistical representation of a morphology of the control segmentations, and wherein the processor segments the anatomical structure of a patient being analyzed for abnormalities to produce a patient segmentation to compare the patient segmentation to the normative dataset obtained from the control segmentations.

A computer-readable storage medium including a set of instructions executable by a processor. The set of instructions operable to segment the anatomical structure imaged in a volumetric image of a plurality of control patients to produce a control segmentation of the anatomical structures of each of the control patients and obtain a normative dataset by extracting a statistical representation of a morphology of the control segmentations.

### BRIEF DESCRIPTION OF THE DRAWINGS

FIG. 1 shows a schematic diagram of a system according to an exemplary embodiment.

FIG. 2 shows a flow diagram of a method according to an exemplary embodiment.

FIG. 3 shows a flow diagram of a method for applying a deformable segmentation, according to the method of FIG. 2.

FIG. 4 shows a perspective view of a deformable brain model according to the method of FIG. 3.

FIG. 5 shows the deformable brain model adapted to a patient's volume according to the method of FIG. 3

### DETAILED DESCRIPTION

The exemplary embodiments may be further understood with reference to the following description and the appended drawings, wherein like elements are referred to with the same reference numerals. The exemplary embodiments relate to a system and a method for identifying volume and shape abnormalities of areas in the brain. In particular, the exemplary embodiments generate a three-dimensional segmentation of patient brain structures, which are adapted to a volumetric image such as an MRI, to compare the segmentations to a normative dataset that includes a quantitative description of the volume and shape of brain structures in healthy individuals. It will be understood by those of skill in the art, however, that although the exemplary embodiments specifically describe the segmentation of brain structures, the systems and methods in the exemplary embodiments may be used to identify volume and shape abnormalities in any anatomical 3D structure in a volumetric image such as, for example, a CT and/or an ultrasound image.

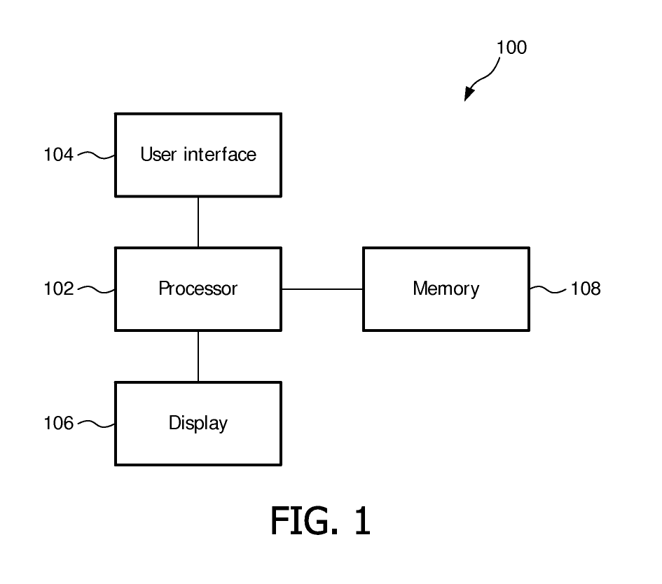

As shown in FIG. 1, a system 100 according to an exemplary embodiment compares a segmentation of a 3D brain structure of interest to a normative dataset to identify volume and shape abnormalities of specific brain areas. The system 100 comprises a processor 102 that is capable of adapting a deformable model of a brain structure based on features of the structure in the volumetric image to both obtain a normative dataset by applying the deformable segmentation to a set of control patients and to a patient whose brain structure is to be analyzed. The processor 102 then compares the segmentation of the brain structure of interest of the patient with the obtained normative dataset of the control patients to identify any abnormalities. The deformable model is selected from a database of models stored in a memory 108. The memory 108 also stores the obtained normative dataset and any segmentations of patient brain structures. A user interface 104 is utilized to input user preferences to determine a volume of the brain structures, view a particular portion of the brain structure, etc. The user interface 104 may be, for example, a graphical user interface displayed on the display 106. Inputs associated with the user interface are entered via, for example, a mouse, a touch display and/or keyboard. The segmentation of the brain structures, the volumetric image and user options of the user interface 104 are displayed on a display 106. The memory 108 may be any known type of computer-readable storage medium.

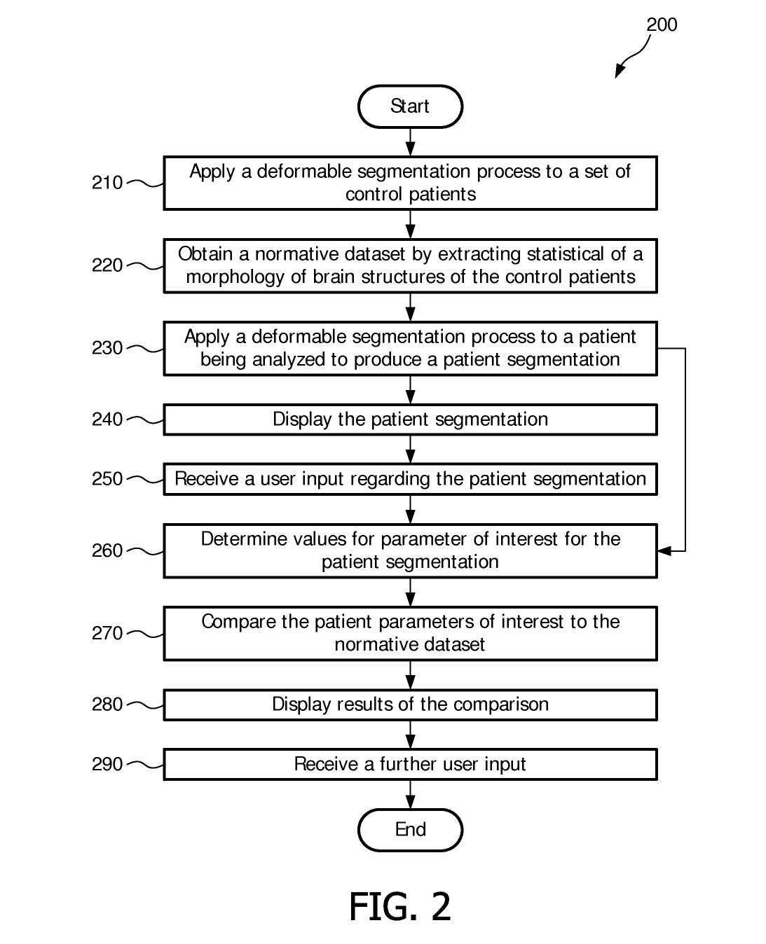

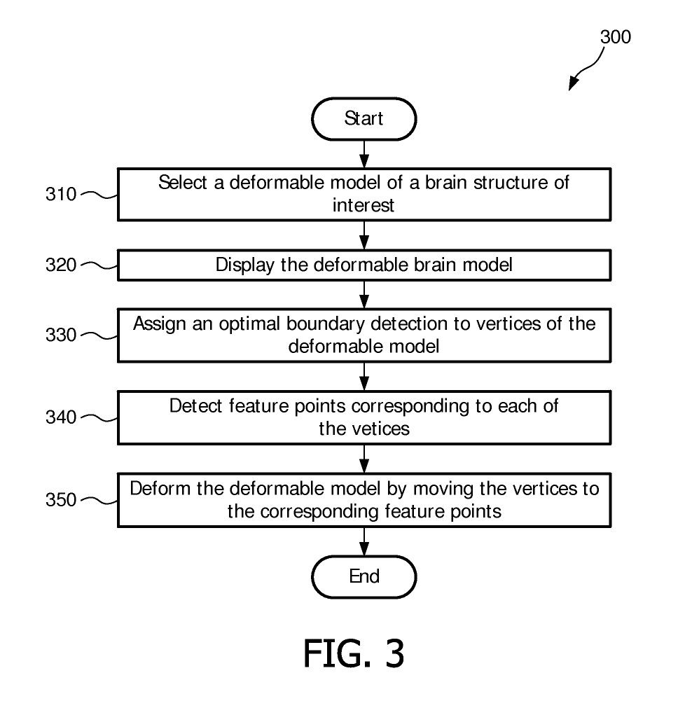

FIG. 2 shows a method 200 according to an exemplary embodiment in which the system 100 compares a 3D patient segmentation of a brain structures of interest to a normative dataset including quantitative information corresponding to the same structure obtained from a group of control patients. The method 200 includes, applying a deformable segmentation process 300 to a set of healthy, control patients, in a step 210, to produce a control segmentation of a brain structure of interest of each of the control patients. It will be understood by those of skill in the art that there may be more than one brain structure of interest and that all of the brain structures may be segmented as described. A detailed description of an exemplary embodiment of the deformable segmentation process 300 is provided below in regard to FIG. 3. In particular, a deformable model of the brain structure is selected and automatically adapted to correspond, in volume and shape, to the brain structures of the control patients.

In a step 220, a normative dataset is obtained based on the deformable segmentation of the structures of the control patients, by extracting a statistical representation of the underlying morphology of the brain structures. The normative dataset will contain information pertaining to volume, shape and a quantitative description of a relationship between different brain structures in the healthy control patient(s), e.g., a statistical description based on mean and variance and/or range values. Complementary to MRI volumes, surfaces representing different brain structures can be used to describe the geometry of the structure exterior. For example, coordinates, voxel values and different shape descriptors (e.g., surface curvature, point displacements from mid-sagittal plane, local deformation of surface, etc.) provide a simple, quantitative description of the brain structure.

Descriptive portions of the normative dataset may further include tags, which may be selected by a user to display textual information regarding the brain structures. The textual information may correspond to other sources such as, for example, radiology reports, that may provide a more complete representation of the normative dataset. Thus, the tags permit variances, biases of the normative dataset to also be compared to a deformable segmentation of brain structures of a patient. It will be understood by those of skill in the art that the normative dataset is stored in the memory 108 such that the normative dataset may be utilized, as desired, for different patients at different times. It will also be understood by those of skill in the art that once the normative dataset has been obtained and stored in the memory 108, the normative dataset may be utilized at any time such that steps 230-290, as described below, may be initiated separately from the steps 210 and 220, as described above.

In a step 230, the deformable segmentation process 300 is applied to a patient whose brain structures are being analyzed to identify abnormalities, to produce a patient segmentation of the brain structure(s) of interest. The deformable segmentation process 300 for the patient is substantially similar to the method of deformable brain segmentation conducted on the healthy control patients in the step 210 and as described below in regard to FIG. 3. In a step 240, the patient segmentation produced in the step 230 is displayed on the display 106. The system 100 then receives a user input, in a step 250, via the user interface 104, which may display user options. The user may enter the user input, electing to store the patient segmentation, retrieve a previously stored patient segmentation, elect to identify abnormalities in the patient segmentation, etc. Other user inputs may include electing to enlarge and/or zoom into a particular portion of the displayed images, change a view of a particular image, etc.

Where the user elects to identify abnormalities via the user input in the step 250, the processor determines values for parameters of interest related to, for example, a volume, shape, curvature and structure of the patient segmentation, in a step 260. The parameters of interest correspond to the types of data included in the normative dataset obtained in the step 220. In a step 270, the values of the parameters of interest of the patient segmentation are compared to the normative dataset obtained from the control segmentations. For example, coordinates, voxel values and other quantitative shape descriptors from the patient segmentation are compared to the values of the normative dataset obtained from the control segmentation. The brain structures of the patient segmentation may be compared individually, as selected by the user, or in the alternative, simultaneously, such that all of the segmented brain structures are analyzed at once. If statistical information is implied within the normative dataset it is possible to directly derive a probability measure of whether or not the structure of interest of the patient's brain is healthy.

In a step 280, results of the comparison between the patient segmentation and the normative dataset obtained from the control segmentation is displayed on the display 106. The displayed results of the comparison may be textual and/or visual. For example, the display 106 may list patient brain structures with identified abnormalities along with a description of the abnormalities. Alternatively, the display 106 may show the patient segmentation with visual indications indicating deviations and/or differences from normative dataset. The visual indications may be, for example, variations in color or color gradients, which can indicate a degree or level of deviation of the patient segmentation from the control segmentation. Different colors may be assigned deviation ranges. Alternatively, the color indications may exist as a color gradient such that levels of deviations are indicated by varying shades of a color.

In a step 290, the system 100 receives a user input via the user interface 104. The user may enter the user input, electing to store the patient segmentation along with comparison results, retrieve a previously stored patient segmentation, select a tag to view, indicate other user preferences, etc. It will be understood by those of skill in the art that although the method 200, as described above, shows that the user elects to compare the patient segmentation to the normative dataset via the user input in the step 250, the comparison may also be conducted automatically by the processor 102 immediately subsequent to the production of the patient segmentation. Thus, it will also be understood by those of skill in the art that the method 200 may also proceed directly from step 230 to the step 260.

FIG. 3 shows an exemplary embodiment of the deformable segmentation process 300, as described above in regard to steps 210 and 230. The method 300 comprises selecting a deformable model of the brain structure of interest from a database of structure models stored in the memory 108, in a step 310. In an exemplary embodiment, the deformable model is automatically selected by the processor 102 by comparing features of the brain structure of interest in the volumetric image to the structure models in the database. In another exemplary embodiment, the deformable model is manually selected by the user browsing through the database to identify the deformable model that most closely resembles the brain structure of interest. The database of structure models may include structure models from brain structure studies and/or segmentation results from previous patients.

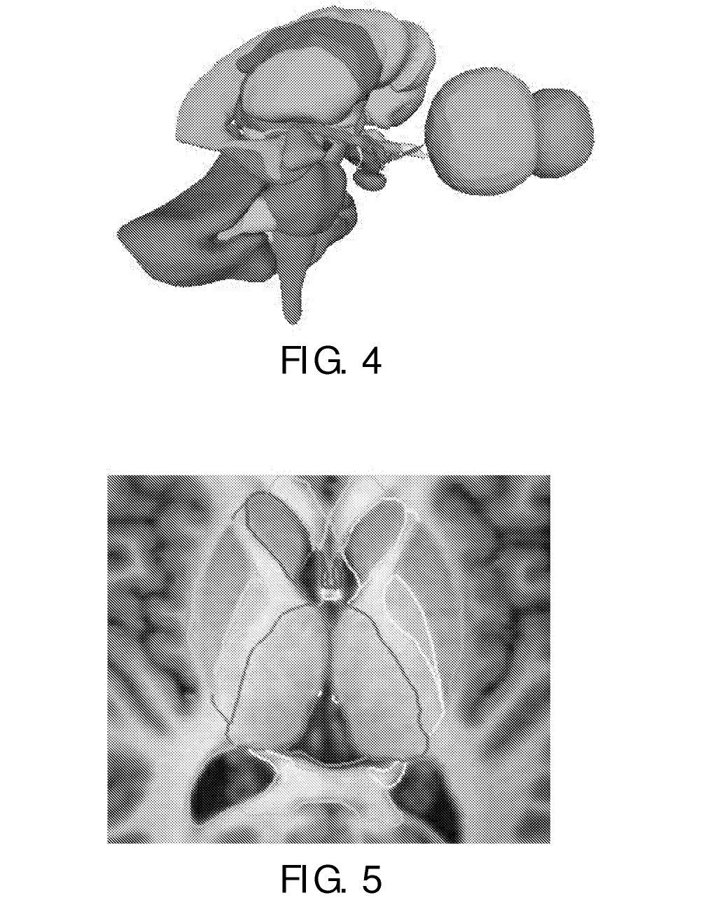

In a step 320, the deformable model is displayed on the display 106, as shown in FIG. 4. The deformable model is displayed as a new image and/or displayed over the volumetric image. The deformable model is formed of a surface mesh including a plurality of triangularly shaped polygons, each triangularly shaped polygon further including three vertices and edges. It will be understood by those of skill in the art, however, that the surface mesh may include polygons of other shapes. The deformable model is positioned such that the vertices of the deformable model are positioned as closely as possible to a boundary of the structure of interest. In a step 330, each of the triangular polygons is assigned an optimal boundary detection function. The optimal boundary detection function detects feature points along a boundary of the structure of interest so that each of the triangular polygons is associated with a feature point, in a step 340. The feature points may be associated with centers of each of the triangular polygons. The feature point associated with each of the triangular polygons may be the feature point that is closest to the triangular polygon and/or corresponds to the triangular polygon in position.

In a step 350, each of the triangular polygons associated with a feature point is automatically moved toward the associated feature point such that vertices of each of the triangular polygons are moved toward the boundary of the structure of interest, deforming the deformable model to adapt to the structure of the interest in the volumetric image. The deformable model is deformed until a position of each of the triangular polygons corresponds to a position of the associated feature point and/or the vertices of the triangular polygon lie substantially along the boundary of the structure of interest, as shown in FIG. 5. Once the deformable model has deformed such that the triangular polygons correspond to the associated feature points of the boundary of the structure of interest, the deformable model has been adapted to the structure of interest such that the deformed deformable model represents a segmented structure of the structure of interest.

It will be apparent to those skilled in the art that various modifications may be made to the disclosed exemplary embodiments and methods and alternatives without departing from the spirit or the scope of the spirit or the scope of the disclosure. Thus, it is intended that the present disclosure cover modifications and variations provided that they come within the scope of the appended drawings and their equivalents.

What is claimed is:

1. A method for identifying an abnormality of an anatomical structure, comprising:
segmenting (210), using a processor (102), the anatomical structure imaged in a volumetric image of a plurality of control patients to produce a control segmentation of the anatomical structures of each of the control patients;
obtaining (220) a normative dataset by extracting a statistical representation of a morphology of the control segmentations;
segmenting (230) the anatomical structure of a patient being analyzed for abnormalities to produce a patient segmentation; and
comparing (270) the patient segmentation to the normative dataset obtained from the control segmentations.

2. The method of claim 1, wherein comparing (270) the patient segmentation includes determining parameters of interest corresponding to a data type of the normative dataset.

3. The method of claim 1, further comprising:
displaying (280) on a display (106) the patient segmentation and results of the comparison between the patient segmentation and the normative dataset via one of textual and a visual indication.

4. The method of claim 3, wherein the visual indications shows a deviation range of the parameters of interest of the patient segmentation from the normative dataset of the control patients via at least one of a color and a color gradient.

5. The method of claim 1, wherein segmenting (230) the anatomical structure further comprises:
selecting (310) a deformable model of the anatomical structure, the deformable model formed of a plurality of polygons including vertices and edges;
displaying (320) the deformable model on a display;
detecting (340) a feature point of the anatomical structure of interest corresponding to each of the plurality of polygons, wherein the feature point is a point substantially along a boundary of the anatomical structure of interest; and
adapting (350) the deformable model by moving each of the vertices toward the corresponding feature points until the deformable model morphs to a boundary of the anatomical structure of interest, forming a segmentation of the anatomical structure of interest.

6. The method of claim 1, wherein the normative dataset includes quantitative values corresponding to at least one a volume and a shape of the control segmentations.

7. The method of claim 6, wherein the quantitative values include a value corresponding to at least one of a surface curvature, a displacement from a mid-sagittal plane and a local deformation of a surface of the control segmentations.

8. The method of claim 1, further comprising:
storing the normative dataset in a memory to be recalled and compared to a patient segmentation.

9. The method of claim 1, further comprising:
receiving (250) a user input regarding the patient segmentation.

10. A system (100) for identifying abnormalities of an anatomical structure, comprising:
a processor (102) segmenting the anatomical structure imaged in a volumetric image of a plurality of control patients to produce a control segmentation of the anatomical structures of each of the control patients and obtaining a normative dataset by extracting a statistical representation of a morphology of the control segmentations, and
wherein the processor (102) segments the anatomical structure of a patient being analyzed for abnormalities to produce a patient segmentation to compare the patient segmentation to the normative dataset obtained from the control segmentations.

11. The system of claim 10, wherein the processor (102) determines values of parameters of interest corresponding to a data type of the normative dataset to compare the patient segmentation to the normative dataset.

12. The system of claim 10, further comprising:
a display (106) displaying the patient segmentation and results of the comparison between the patient segmentation and the normative dataset via one of textual and a visual indication.

13. The system of claim 12, wherein the visual indications shows a deviation range of the parameters of interest of the patient segmentation from the normative dataset of the control patients via at least one of a color and a color gradient.

14. The system of claim 10, wherein segmenting the anatomical structure includes the processor (102) selecting a deformable model of the anatomical structure, the deformable model formed of a plurality of polygons including vertices and edges,
wherein the display (106) displays the deformable model,
wherein the processor (102) further detects a feature point of the anatomical structure of interest corresponding to each of the plurality of polygons and adapts the deformable model by moving each of the vertices toward the corresponding feature points until the deformable model morphs to a boundary of the anatomical structure of interest, forming a segmentation of the anatomical structure of interest, and
wherein the feature point is a point substantially along a boundary of the anatomical structure of interest.

15. The system of claim 10, wherein the normative dataset includes quantitative values corresponding to at least one a volume and a shape of the control segmentations.

16. The system of claim 15, wherein the quantitative values include a value corresponding to at least one of a surface curvature, a displacement from a mid-sagittal plane and a local deformation of a surface of the control segmentations.

17. The system of claim 10, further comprising:
a memory (108) storing the normative dataset to be recalled and compared to a patient segmentation.

18. The system of claim 10, further comprising:
a user interface (104) receiving user inputs regarding the patient segmentation.

19. A computer-readable storage medium (108) including a set of instructions executable by a processor (102), the set of instructions operable to:
segment (210) the anatomical structure imaged in a volumetric image of a plurality of control patients to produce a control segmentation of the anatomical structures of each of the control patients; and
obtain (220) a normative dataset by extracting a statistical representation of a morphology of the control segmentations.
